(19) United States
(12) Patent Application Publication
(10) Pub. No.: US 2012/0245529 A1
(43) Pub. Date: Sep. 27, 2012

(54) ANCHORING DEVICE FOR A LINE IN A SKULL BORE HOLE

(75) Inventors: Jörg Hummen, Kilchberg (CH); Cyrill Röthlin, Hunenberg (CH); Claudio Steiner, Schwyz (CH)

(73) Assignee: CARAG AG, BAAR (CH)

(21) Appl. No.: 13/514,002

(22) PCT Filed: Dec. 2, 2010
(86) PCT No.: PCT/CH2010/000306

§ 371 Date: Jun. 5, 2012

(30) Foreign Application Priority Data
Dec. 11, 2009 (CH) 1907/09

(51) Int. Cl. A61M 25/04

(52) U.S. Cl. 604/175

(57) ABSTRACT

An anchoring device for anchoring a line in a skull bore hole comprises a base part and a cover that tightly closes the base part. The base part has a fastening element for fastening the base part in the bore hole and a passage opening for feeding the line from the bore hole to an outside of the anchoring device. A contact surface extending between the bore hole and the outside for laying the line is present in the base part. The contact surface is curved in a constant direction so that the line is guided from the skull interior to the outside in a constant direction and at a radius of curvature that is greater than or equal to a predefined minimum radius of curvature. Said anchoring device allows a line to be lead out of a bore hole of a skull at a flat angle without the line being bent too greatly or even kinked.

### CROSS REFERENCE TO RELATED APPLICATIONS

The present application is a U.S. National Phase Application pursuant to 35 U.S.C. §371 of International Application No. PCT/CH/2010/000306 filed Dec. 2, 2010, which claims priority to Swiss Patent Application No. 1907/09 filed on Dec. 11, 2009. The entire disclosure contents of these applications are herewith incorporated by reference into the present application.

### TECHNICAL FIELD

The present invention relates to an anchoring device for anchoring a line in a skull bore hole.

### PRIOR ART

The prior art has disclosed various methods and means for cerebral diagnostics and/or therapy, which require a line through a bore hole of a skull. Examples of these are devices for the electrical brain stimulation, for measuring the perfusion or the blood flow, for pressure measurements, for supplying medicaments and for the aspiration of tissue liquid.

Here, the line is fixed or anchored in the bore hole in order to avoid inadvertent injury to the brain. In particular, movements in the longitudinal direction of the line should be avoided. For this purpose, use is preferably made of compression fittings that can be screwed. The prior art has disclosed various anchoring devices. There are devices that are arranged under the scalp. In this case, the line generally still runs a specific distance along the skull between skull and scalp before it emerges from the scalp. In other applications, the anchoring device penetrates the scalp. The line is then usually adhesively bonded to the scalp using appropriate adhesive strips.

By way of example, such anchoring devices have been disclosed in U.S. Pat. No. 6,923,799, U.S. Pat. No. 4,677,985 and U.S. Pat. No. 7,553,290. U.S. Pat. No. 4,328,813 describes that a line is clamped between a base part and a sealing cover, and is thus routed out of the skull at a relatively small and flat angle with respect thereto. U.S. Pat. No. 6,044,304 describes an annular base part with a central perforated plate, through the holes of which the line can be routed out. The annular body has recesses running in the radial direction such that the lines routed to the outside run at an angle to the skull which is as small as possible. U.S. Pat. No. 5,843,150 shows a base part and a cover which is open to the top. Laterally, the cover has a passage opening for routing through the line. U.S. Pat. No. 5,927,277 uses a clamping-ring insert in the base part and the line is routed out of the skull through a lateral recess in the base part. In this case, the opening is sealed with a plug. US 2005/0182464 discloses further anchoring devices, which have relatively complicated designs.

### DESCRIPTION OF THE INVENTION

It is therefore an object of the invention to create an anchoring device for anchoring or fixing a line in a skull bore hole, which device has a relatively simple design and makes it possible to route the line out of the bore hole as safely as possible and at an angle which is as flat as possible.

The anchoring device according to the invention for anchoring a line in a skull bore hole has a base part. In addition it can comprise a cover which securely seals the base part. The base part comprises a fixing element for fixing the base part in the bore hole and a passage opening for routing the line therethrough, out of the bore hole to an external side of the anchoring device. A contact face for positioning the line and extending between bore hole and external side is present in the base part. The contact face has a curved embodiment in an unchanging direction such that the line is routed from the interior of the skull to the external side with an unchanging direction and with a radius of curvature, which is equal to or greater than a predefined minimal radius of curvature. The curvature is preferably continuous, i.e. stepless, and it is preferably curved toward the longitudinal central axis of the device. The whole arrangement is preferably aligned centrally with respect to the longitudinal central axis of the device.

The device according to the invention not only fixes the line, but it also prevents said line from being squeezed or kinking Since the lines cannot be bent too strongly and hence squeezing or kinking is made impossible, the functionality thereof is ensured. This is important, particularly in the case of drainage tubes and optical-fiber lines.

The lines can nevertheless be routed out of the anchoring device close to the scalp or the skull. As a result of this flat angle, the risk of injury to the brain of the patient is minimized.

In the following text, use is made of the term “anchoring device”. The same device is also referred to as “port” or “lock”. Depending on field of application, a line is understood to mean a probe, a catheter, a drainage tube, an electrode or a differently embodied line.

In a preferred embodiment there is a passage channel for holding the line, which passage channel is formed firstly by the contact face and secondly by the cover. The cover preferably closes the base part in an interlocking manner except for the formation of the passage channel. To this end, it preferably has an inner surface which is matched to the contact face of the base part and presses the line against the contact face. There preferably is a radial groove in the base part, into which groove the line can be inserted. In the groove 22, the line is preferably held in a clamped and interlocking manner. However, as an alternative or in addition thereto, a corresponding groove can be provided in the cover.

In a preferred embodiment, the line is also supported by the base part outside of the passage channel and routed onto the skull or the scalp at a relatively flat angle and without kinks By way of example, this can be achieved by at least one lug which protrudes radially outward. This lug has an upper side, which adjoins the curved contact face in continuous fashion and hence in a stepless fashion and serves as external contact face, tilted toward the skull, for the line. If a number of lugs are present, the line can be routed out of the base part at an optimized side without the base part needing to be brought into another rotational position for this purpose. This embodiment can also be used without curved internal region, i.e. without inner cone.

The fixing element preferably has a male thread which can be screwed into the bore hole. This male thread preferably has a self-cutting embodiment. It can be made easier to screw in the base part if there are a plurality of lugs, more particularly three lugs, which are arranged equidistantly over the external circumference of the base part. These lugs serve as turning handles for screwing the base part into the bore hole. They can be identical to the aforementioned lugs which serve as contact face for the line or they can be formed by further lugs.

In a preferred embodiment, the passage opening has a funnel, wherein the funnel tapers toward the bore hole and wherein the inner side of the funnel has an arced embodiment toward the longitudinal central axis of the funnel, more particularly a convex embodiment. The cover preferably also has a conical sealing body which projects toward the base part and hence forms an interlocking connection in this region.

The base part can, in part or overall, have an integral design. In particular, at least the base body and the insert element can have an integral design and the seal can form a separate part. However, it preferably has a multipart design. By way of example, it can comprise an annular body with a central passage opening and an insert element held in the central passage opening of the annular body, wherein the insert element forms the funnel.

So that the base element cannot be screwed too far into the bore hole and therefore is prevented from applying pressure to the scalp and brain structures, there preferably is a neck on the fixing element, which neck has a greater diameter than the male thread and is arranged between male thread and remaining base part.

An annular seal is preferably present for fixing the line in the base part in sealing fashion. If the annular seal is embodied with an obliquely protruding, resilient skirt, the line can easily be pushed in the direction of the skull interior, but it is very difficult to pull it out.

The cover seals the base part and hence the bore hole. It forms protection from bacteria or protection against inward migration of microorganisms and/or dirt particles over a period of a number of days and weeks. In order to optimize this protection, it is possible to introduce antibacterial means, e.g. a gel, into the passage opening, more particularly into the funnel of the base part and/or the grooves. It is also possible to apply an antibacterial coating, for example with silver ions, to the inner side of the cover.

By means of the seal, more particularly the circular sealing lips, and an antimicrobial substance, more particularly in gel, arranged thereover, the device according to the invention provides for double containment against inward migration of microorganisms and/or dirt particles.

If the cover is provided with flanges or straps which protrude radially outward, it can be attached to a scalp or the skull. In particular, it can be sewn onto the scalp.

Further embodiments are specified in the dependent claims.

### BRIEF DESCRIPTION OF THE DRAWINGS

Preferred embodiments of the invention are described in the following text on the basis of the drawings, which merely serve for explanatory purposes and should not be construed as restrictive. In the drawings:

FIG. 1 shows an exploded view of an anchoring device according to the invention in a perspective view as per a first embodiment;

FIG. 2 shows a partial exploded view of the anchoring device as per FIG. 1 in a partial section;

FIG. 3 shows a longitudinal section through the anchoring device as per FIG. 1 in the assembled state;

FIG. 4 shows a view of a cover of the anchoring device as per FIG. 1 obliquely from below;

FIG. 5 shows a view of the anchoring device as per FIG. 3, assembled as intended, from a first side;

FIG. 6 shows a view of the anchoring device as per FIG. 5, from a second side;

FIG. 7 shows a perspective view of a base part according to the invention in a second embodiment;

FIG. 8 shows a perspective view of the base part as per FIG. 7 with a cover according to the invention as per this second embodiment, from a first side;

FIG. 9 shows a perspective view of the anchoring device as per FIG. 8, from a second side;

FIG. 10 shows a perspective view of the cover as per FIG. 9, from below;

FIG. 11 shows a partial section in an exploded view of the anchoring device according to the invention as per FIG. 8, from a first side; and

FIG. 12 shows a partial section of the anchoring device as per FIG. 11, from a second side.

### DESCRIPTION OF PREFERRED EMBODIMENTS

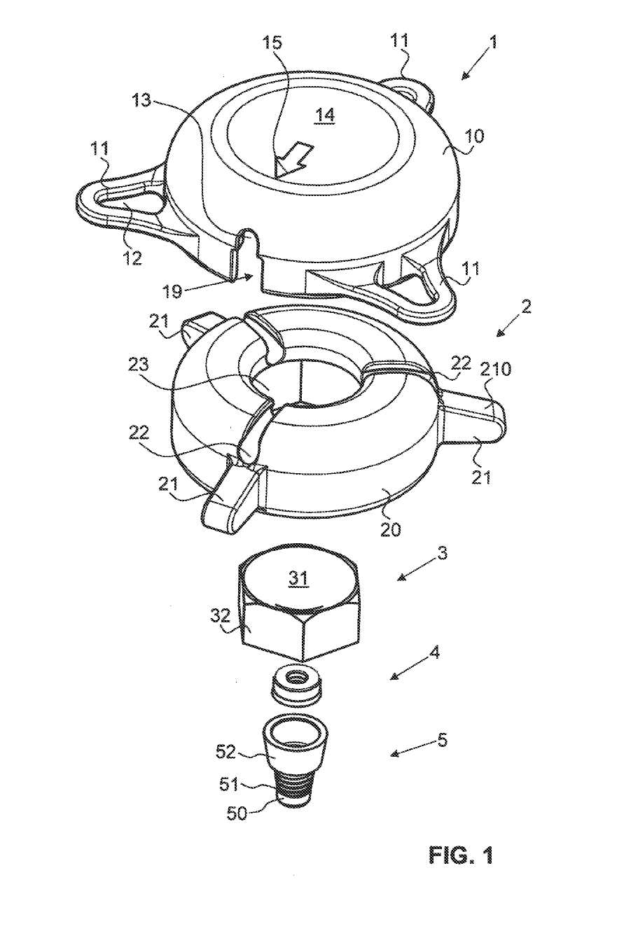

FIG. 1 shows an exploded view of a preferred exemplary embodiment of the anchoring device according to the invention. It consists of at least two parts, a cover 1 and a base part 2, 3, 4, 5. The base part can have an integral design or, as illustrated here, a multipart design. In this example, the base part consists of an annular body 2, an insert element 3, a seal 4 and a fixing element 5. The cover 1 preferably has an integral design.

The cover 1 is preferably produced from an elastic, or at least soft, plastic, for example silicone. The annular body 2 preferably consists of a hard plastic, for example polycarbonate. Insert element 3 and fixing element 5 are preferably manufactured from metal or plastic, for example titanium or polycarbonate. The seal 4 preferably consists of silicone.

The cover 1 has a base body 10, which has an approximately planar cover face 14 and an approximately cylindrical lateral shell 17. The transition region 18 between cover face 14 and lateral shell 17 has a curved embodiment. Attachment elements 21 protrude radially from the lateral shell 17. In this case, three attachment elements are arranged distributed uniformly over the circumference. A different number, for example one, two, four or five, is also possible. In this example, the attachment elements are formed by straps 11, which each surround one attachment hole 12. The straps 11 are arranged on the lower side of the lateral cover shell 17, which faces the skull in the implanted state. Furthermore, the lateral shell 17 has recesses 19 (see FIG. 4), which are open downward, toward the skull. These are preferably arranged centrally between the straps 11 and are preferably equal in number to the straps, but in any case are equal in number to the lugs 21, mentioned below, of the base part 2. One of these recesses 19 is extended at the top by an inlet opening 13. There preferably is a marking 15, in this case an arrow, on the cover face 14 such that the position of the passage opening 13 is visible from above, i.e. from the cover face. The inlet opening 13 is preferably arranged centrally between two attachment elements 11.

An inwardly protruding sealing body 16 is formed on the cover face 14 on the inner side of the cover 1. This sealing body 16 tapers downward and forms a taper, i.e. a cone or a frustum, with curved lateral face or curved flanks. The lateral face preferably has an inwardly curved embodiment.

The annular body 2 has an annular base body 20, which surrounds a central passage opening 23. Its lower side 24 facing the skull preferably has a concave embodiment. Its upper cover face 27 preferably has a planar embodiment and its lateral shell 25 is substantially cylindrical. The transition region 26 between cover face 27 and lateral shell 25 likewise has a curved embodiment. In terms of shape and dimensions, the external basic shape of the annular body 2, i.e. the cover face 27, the lateral shell 25 and the transition region 26, corresponds to the shape and the dimensions of the inner side of the cover 1. The outer cross-sectional shape of the lugs also preferably corresponds to the shape of the recesses 19. However, the dimensions of these parts of the annular body 2 are preferably slightly larger so that the cover 1, when it is placed over the annular body 2, lies in sealing fashion against the cover face 27, the transition region 26 and the lateral shell 25 of the annular body 2 as a result of the elastic material thereof.

The annular body 2 furthermore has lugs 21 which radially protrude from the lateral shell 25. The face 210 of the lugs 21 directed upward, i.e. toward the cover 1, has an embodiment that drops radially outward. The face 220 directed downward, i.e. toward the skull, preferably has an embodiment that is level such that the thickness of the lugs 21 decreases radially outwardly. Three such lugs 21 preferably are arranged equidistantly over the circumference of the annular body 2. However, only one, two, four, five or more lugs 21 may also be present. It is preferable for the number of lugs 21 to equal the number of straps 11. The lugs 21 are preferably formed onto the lateral shell 25 and are situated in the lower region thereof, but preferably at a distance from the lower edge of the annular body 2.

The annular body 2 moreover also has grooves 22 which run in the radial direction, which grooves connect the central passage opening 23 with the external side of the annular body 2. These grooves 22 preferably end at the lugs 21 such that these respectively extend along the same radius. Toward the outside, the bases of the grooves 22 are likewise embodied tilting downward, wherein they preferably merge in a stepless fashion, i.e. continuously, into the surface of the lugs 21. As a result there are stepless and continuous channels between the central passage opening 23 and the external side of the annular body 2. The lugs 21 preferably extend up to the upper end of the lateral shell 25 of the annular body 2, or they end just therebelow.

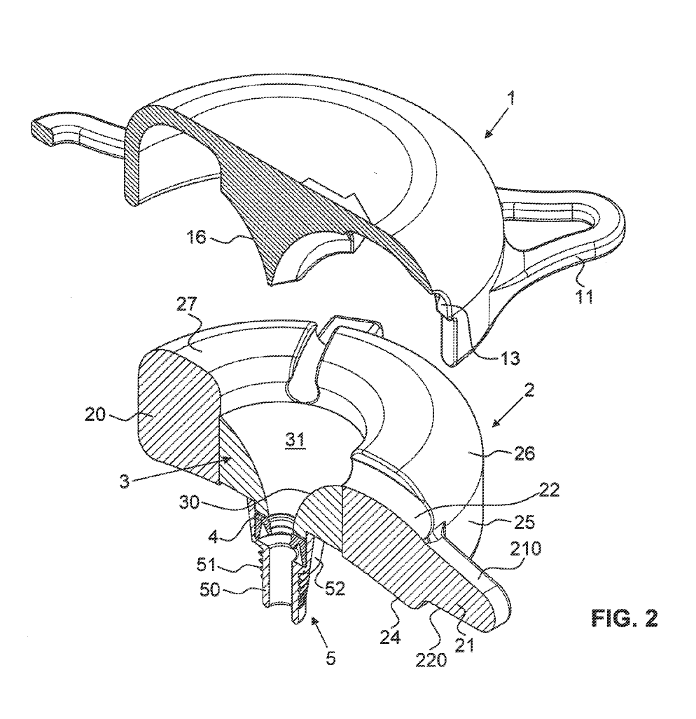

The insert element 3, the seal 4 and the fixing element 5 are clearly identifiable in FIG. 2. They have already been connected to the annular body 2 in this FIG. 2. The insert element 3 is arranged in the central passage opening 23, wherein it is preferably pressed therein. For this purpose, the external side of the insert element 3 and the wall of the central passage opening 23 both preferably have the shape of a hexagon, as can be seen in FIG. 1. However, use can also be made of any other shapes.

The insert element 3 has a tilted inner surface, which tapers downward, i.e. toward the skull. As a result, this creates a central funnel-shaped opening with preferably curved flanks 30. If the base part is assembled as intended, the upper edge of this flank 30 or this funnel is flush with radially running grooves 22. This upper edge forms a stepless and hence continuous transition to the base of these grooves 22.

Hence the insert element 3 has a large upper passage opening and a small lower passage opening, which are interconnected by a curved region. The lower opening of the insert element 3 is inserted into the fixing element 5, wherein the seal 4 is also held in this connection region. There are various options for fixing these three parts 3, 4, 5 relative to one another. By way of example, the seal 4 can be inserted into the fixing element 5, these two parts 4, 5 can together be pressed and fused into the insert element 3 and these three parts 3, 4, 5 can be pressed into the annular body 2.

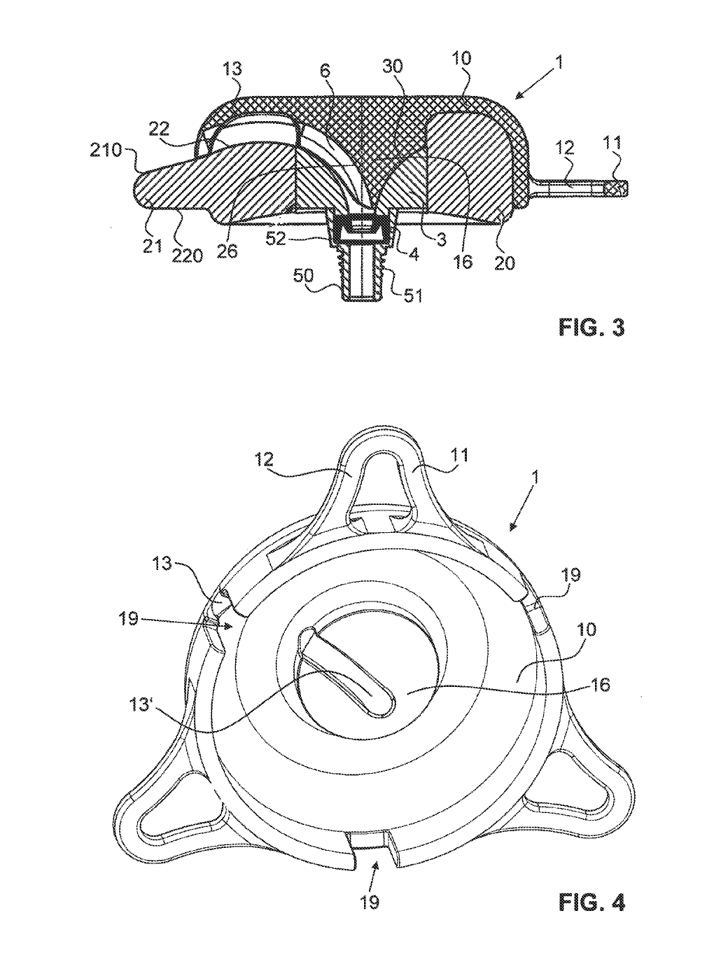

It is clearly visible in FIGS. 2 and 3 that the seal 4 preferably is an annular seal. It preferably has a resilient design. In this embodiment, it consists of two encircling sealing lips, which form an angle. Here, it has an encircling sealing lip which is directed toward the longitudinal axis of the arrangement and hence protrudes into the lower passage opening of the insert element 3. With a second sealing lip, said encircling sealing lip contacts an approximately cylindrical inner face of the fixing element 5. This seal fixes the line in its position within the fixing element. Due to the shape thereof, it enables a simple insertion of the line, and makes pulling said line out more difficult. The seal 4 forms protection against the inward migration of microorganisms and/or dirt particles into the skull.

The fixing element 5 has a plug-in body 50, which for example can have a hollow cylindrical embodiment. However, as illustrated here, it is preferably embodied like a frustum with a downwardly tapering diameter and an axial passage opening. This conical shape is advantageous in that splinters created when the fixing element 5 is screwed into the bore hole are transported outward and cannot reach the brain structures.

The plug-in body 50 has a male thread 51 over at least part of the axial length thereof. The upper neck 52, facing the annular body 2, of the fixing element 5 preferably has a wider diameter than the plug-in body 50, with the transition being embodied as a step. This neck 52 preferably likewise has a frustum-like embodiment, with its diameter also increasing upward toward the annular body 2.

In FIG. 3, the anchoring device according to the invention is illustrated in the state where it is assembled as intended. The cover 1 is placed over the annular body 2, wherein the inlet opening 13 lies over one of the lugs 21 and forms part of a passage channel 6 with one of the grooves 22. This passage channel 6 extends from the lower end face of the fixing element 5, via the central lower opening of the insert element 3, via the curved flanks 30 of this insert element 3 up to the external side of the annular body 2. As can be identified in FIG. 3, the cover 1 closes off the remaining regions and, in particular, the flanks 30 of the insert element 3 in sealing fashion.

FIG. 4 shows the cover 1 in a view obliquely from below. It is possible to identify here that the inlet opening 13, corresponding to the shape of the inner side of the cover 1, extends to the center and hence to the lowest tip of the sealing body 26. In this case, the axial or central end of the inlet opening 13 merges into a channel, open on one side, and has the reference sign 13′.

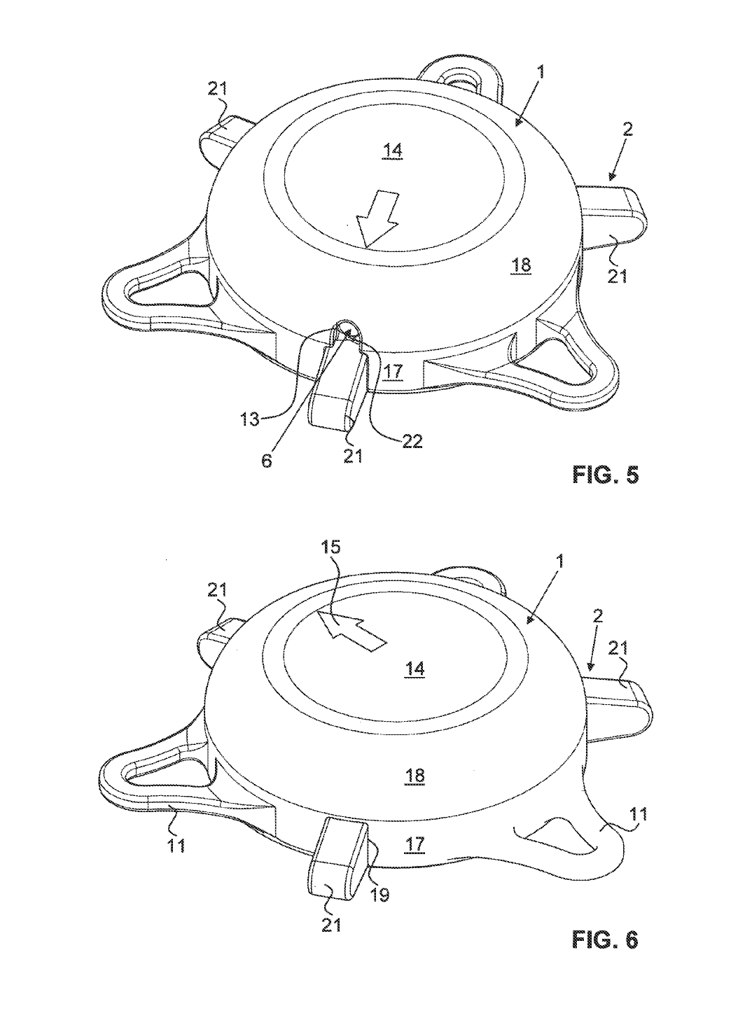

FIG. 5 shows a perspective illustration of the anchoring device, assembled as intended, from a first side, in which the inlet opening 13, arranged over a lug 21, is visible. FIG. 6 shows the same device from another side, with it being possible to identify that the other lug 21 is covered in sealing fashion by the cover 1 and there is no opening. The third lug 21 is also closed in sealing fashion by the cover 1, although this cannot be identified here.

This anchoring device according to the invention can now be used as follows: After a bore hole was bored into a skull, the base part can be screwed through the scalp into the bore hole by means of the fixing element 5 and thus be fixed in the skull. Here, the protruding lugs 21 of the annular body 2 make the screwing-in of the fixing element 5 easier because they serve as a hold for the hand of the surgeon and thus form a relatively large and well controllable lever when screwing-in the part. The neck 52 serves as spacer between skull and scalp. The length of the neck 52 preferably approximately corresponds to a typical thickness of a scalp. The concave lower face 24 of the annular body 2 is approximately fitted to the shape of the skull. Once the base part has been fixed, a line, more particularly a probe, a catheter, a drainage tube, an electrode or any other type of line, depending on the field of application is now routed through the passage channel 6, which is clearly identifiable in FIGS. 4 and 5. Here, the line is routed through the annular body 2, the insert element 3, the annular seal 4 and the fixing element 5 into the interior of the skull. It lies on the arced cone of the insert element such that this cone serves as contact face. The line is furthermore placed into one of the grooves 22 of the annular body 2, which grooves likewise serve as contact face. The grooves 22 are preferably dimensioned such that the line has to be pressed therein and is held in its position in an interlocking fashion. By way of example, the grooves 22 can have a cross section corresponding to a partial circle, with this partial circle being greater than a semicircle, i.e. the ends of which include an angle of more than 180°.

If the cover 1 is now placed over the annular body 2, the line is fixed in its position in the passage channel 6 and, more particularly, it can no longer move in its longitudinal direction. Due to the seal 4, the interlocking connection in the groove 22, the interlocking connection as a result of the cover 1 and due to the deflection by more than 90°, the line is held in its position. Due to the continuous transitions and also the ramp-shaped, curved faces and the lugs 21 tilting toward the skull, the line is routed at a gentle radius up to the external side of the skull or to the external scalp. The external, i.e. non-subcutaneous, region of the line can thus be attached to the head. In the region of the anchoring device (which is also referred to as a port), the line is not subjected to any tensile or pressure forces and, in particular, there is no kinking This is particularly advantageous in the case of optical-fiber lines, drainage tubes or other lines with a continuous lumen because its admissible minimum bending radius can be taken into account by appropriate shaping of the base part.

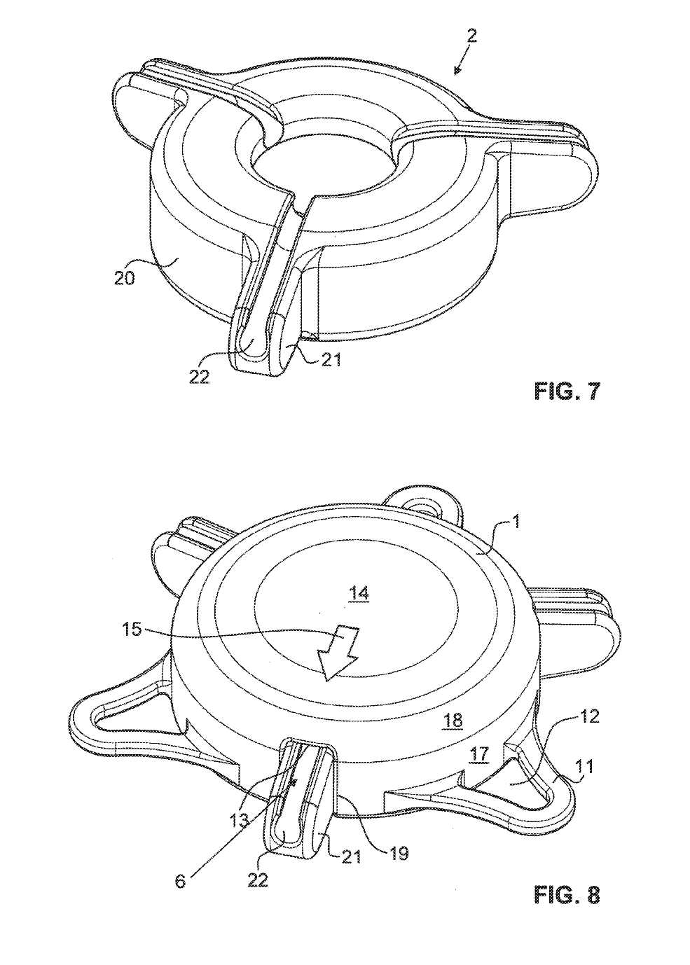

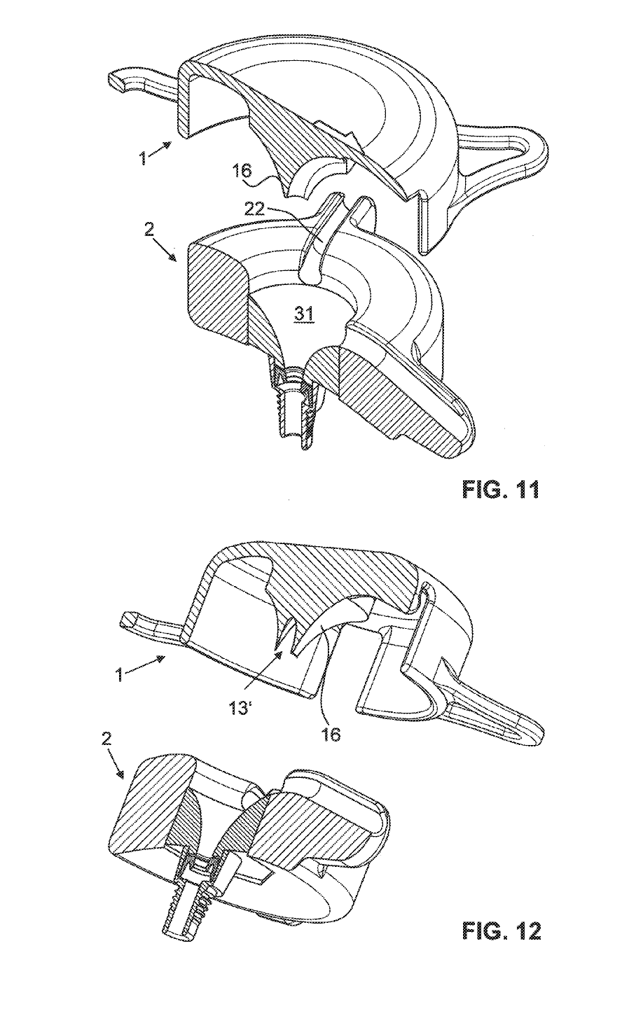

FIGS. 7 to 12 illustrate a second embodiment of the anchoring device according to the invention. Equivalent parts have been provided with the same reference signs. The aforementioned description also applies to this embodiment and so it will not be repeated.

The anchoring device as per this example primarily differs from the first example in that the grooves 22 do not end in the base body 20 but also extend over the lugs 21. This can clearly be identified in FIG. 7 in particular. Here, the transition of the grooves 22 from the base body 20 to the lugs 21 is stepless, i.e. continuous. Here, the groove base increasingly tilts toward the underside 220 of the lug 21, and so the transition of the line, downstream of the outer end of the groove 22, to the skull or the scalp only still has a small step and can therefore occur at a very flat angle.

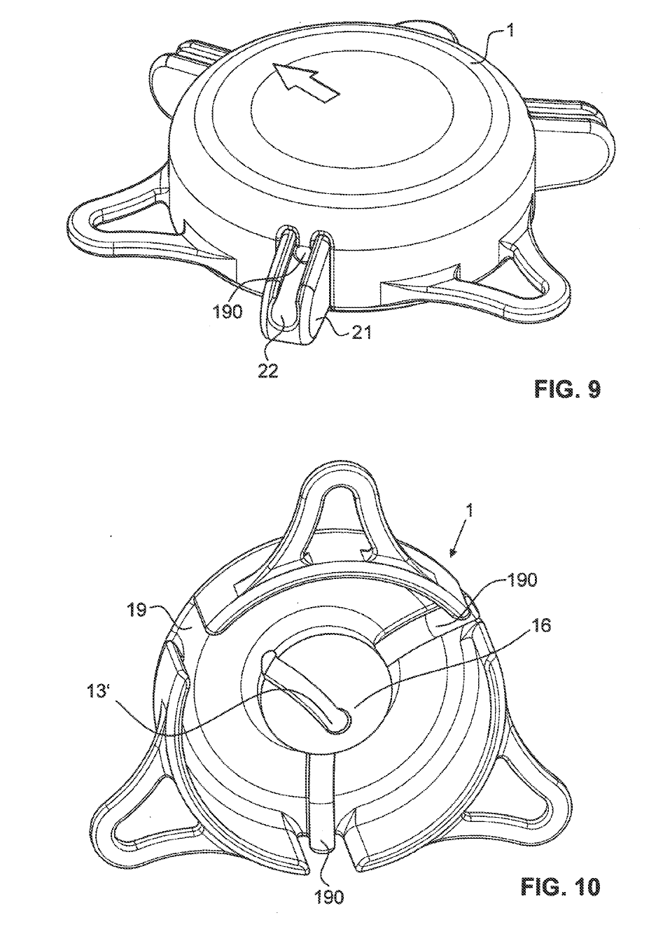

FIGS. 8 and 9 show the base part as per FIG. 7 with a cover 1 placed thereover. The passage channel 6, through which the line is routed to the outside, has an open embodiment since the cover has a corresponding inlet opening 13. It can be identified in FIG. 8. The grooves 22 of the two other lugs 21 are closed to the outside, as can be identified in FIG. 9. To this end, the cover 1 has respectively one radial sealing rib 190. These ribs 190 can clearly be identified in FIG. 10.

The cover 1 preferably seals the funnel of the insert element 3 with a conical sealing body 16 in this example as well, with said cover having a recess in order to enable a passage of the line from the funnel of the insert element 3 to the external side of the anchoring device. This passage is formed by the already mentioned inlet opening 13 or inlet channel. The central end of this inlet opening or of the inlet channel is denoted by 13′ in FIG. 12.

The anchoring device according to the invention makes it possible to route a line out of a bore hole in a skull at a flat angle, without the line being bent too strongly or even being kinked.

1. An anchoring device for anchoring a line in a skull bore hole, having a base part, wherein the base part has a fixing element for fixing the base part in the bore hole and a passage opening for routing the line therethrough, out of the bore hole to an external side of the anchoring device, wherein a contact face for positioning the line and extending between bore hole and external side is present in the base part, wherein the contact face has a curved embodiment in an unchanging direction such that the line is routed from the interior of the skull to the external side with an unchanging direction and with a radius of curvature, which is equal to or greater than a predefined minimal radius of curvature.

2. The anchoring device as claimed in claim 1, wherein there is a passage channel for holding the line, which passage channel is formed firstly by the contact face and secondly by the cover.

3. The anchoring device as claimed in claim 2, wherein the cover closes the base part in an interlocking manner except for the formation of the passage channel.

4. The anchoring device as claimed in claim 1, wherein the base part has at least one lug which protrudes radially outward and has an upper side, wherein this upper side adjoins the curved contact face in continuous fashion and serves as external contact face, tilted toward the skull, for the line.

5. The anchoring device as claimed in claim 4, wherein there are a plurality of lugs, more particularly three lugs, which are arranged equidistantly over the external circumference of the base part and which are embodied as turning handles for screwing the base part into the bore hole.

6. The anchoring device as claimed in claim 1, wherein the cover has an inner surface which is matched to the contact face of the base part and presses the line against the contact face.

7. The anchoring device as claimed in claim 1, wherein the passage opening has a funnel, wherein the funnel tapers toward the bore hole and wherein the inner side of the funnel has an arced embodiment toward a longitudinal central axis of the funnel.

8. The anchoring device as claimed in claim 1, wherein the cover has a conical sealing body which projects toward the base part.

9. The anchoring device as claimed in claim 1, wherein the base part has at least one groove, which runs radially from an inner side to the outside and into which the line can be inserted.

10. The anchoring device as claimed in claim 1, wherein the fixing element has a male thread for fixing the base part in the bore hole and wherein a neck is arranged between male thread and remaining base part, which neck has a greater diameter than the male thread and serves as spacer between skull and remaining base part.

11. The anchoring device as claimed in claim 1, wherein an annular seal is present for fixing the line in the base part in sealing fashion.

12. The anchoring device as claimed in claim 1, wherein protection against inward migration of microorganisms and/or dirt particles has been introduced into the passage opening.

13. The anchoring device as claimed in claim 1, wherein the cover is provided on the inner side thereof with protection against inward migration of microorganisms and/or dirt particles.

14. The anchoring device as claimed in claim 1, wherein the cover has attachment elements which protrude radially outward, by means of which said cover can be attached to a scalp or the skull.

15. An anchoring device for anchoring a line in a skull bore hole, having a base part and a cover which securely seals the base part, wherein the base part has a fixing element for fixing the base part in the bore hole and a passage opening for routing the line therethrough, out of the bore hole to an external side of the anchoring device, characterized in that the base part has at least one groove, which runs radially from an inner side to the outside and has a groove base, and wherein this groove merges into a radially outward protruding lug, wherein this lug has an upper side, which adjoins the groove base of the groove in a stepless fashion and serves as external contact face, tilted toward the skull, for the line.

16. The anchoring device as claimed in claim 1, wherein the anchoring device has a cover which securely seals the base part.

17. The anchoring device as claimed in claim 15, wherein the anchoring device has a cover which securely seals the base part.
